(19) United States
(12) Patent Application Publication
(10) Pub. No.: US 2006/0147443 A1
(43) Pub. Date: Jul. 6, 2006

(54) Synthetic biomaterials having incorporated therein bioactive factors through enzymatically degradable linkages

(75) Inventors: Jason Schense, Zurich (CH); Didier Cowling, Thalwil (CH); Matthias Lutolf, Zurich (CH); Annemie Rehor, Zurich (CH)

Correspondence Address: PATREA L. PABST;PABST PATENT GROUP LLP, 400 COLONY SQUARE, SUITE 1200, ATLANTA, GA, 30361, US

(73) Assignee: Kuros Biosurgery AG

(21) Appl. No.: 11/317,846

(22) Filed: Dec. 22, 2005

Related U.S. Application Data
(60) Provisional application No. 60/638,518, filed on Dec. 22, 2004.

(51) Int. Cl. A61K 38/37; A61K 38/48

(52) U.S. Cl. 424/94.63; 514/12

(57) ABSTRACT

Synthetic biomaterials containing bioactive factors or modified bioactive factors that are covalently bound to the synthetic precursor components and/or biomaterials by an enzymatically degradable linkage are described herein. Further described are methods to covalently bind bioactive factors to synthetic biomaterials by means of enzymatic catalysis, the biomaterials produced therewith and the bioactive factors necessary for practicing these methods. The bioactive factors contain an amino acid sequence which can serve as a substrate domain for cross-linkable enzymes. The enzyme catalyzes the cross-linking reaction between the substrate domain of the bioactive factor and functional groups of the synthetic precursor components capable of forming the biomaterial and/or synthetic biomaterial susceptible to an enzymatically catalyzed cross-linking reaction. The biomaterials described herein may be used for localized delivery of the bioactive factors, for tissue repair and regeneration and in particular for regeneration of soft and hard tissue, such as skin, bone, tendons and cartilage.

### CROSS-REFERENCE TO RELATED APPLICATIONS

This application claims priority to U.S. Ser. No. 60/638,518, filed Dec. 22, 2004.

### FIELD OF THE INVENTION

The present invention relates to synthetic biomaterials with bioactive factors incorporated therein, to a method of binding and release of bioactive factors to and from said biomaterials and to methods for applying and use of said biomaterials supplemented with bioactive factors.

### BACKGROUND OF THE INVENTION

Natural and synthetic biomaterials, like fibrin matrices or synthetic polyethylene-based hydrogels, can be used in a variety of applications, including pharmaceutical and surgical applications. They can be used, for example, to deliver bioactive factors to a subject, as adhesives or sealants, tissue engineering or wound healing scaffolds, or cell transplant devices.

For application in the human and animal body, in-situ formation of biomaterials at the site of need in the body is the technique of choice since the biomaterial can be applied by minimally invasive surgery. However, the application in the body limits the choice of chemistry with regard to (i) the nature of the precursor components forming the biomaterial, (ii) the cross-linking mechanism for in-situ formation of the biomaterial, and (iii) the cross-linking mechanism for incorporating the bioactive factor to the precursor components and/or biomaterials.

With regard to the precursor components varying approaches have been employed. In one approach, naturally occurring precursor components are utilized; another approach focuses on completely synthetic precursor components; and in still another approach combinations of naturally occurring and synthetic precursor components or modifications of one or the other are used.

Biomaterials based on naturally occurring or chemically modified naturally occurring proteins, like collagen, denatured collagen (gelatin) and in particular fibrin have been applied in human and animal bodies. In particular good responses have been achieved with matrices based on fibrin and collagen. Other examples include carbohydrates, like cellulose, alginates and hyaluronic acid.

The incorporation of bioactive factors in natural or synthetic biomaterials or mixtures thereof are mainly done by incorporation of the bioactive factor through physical interaction as has been described, for example, in U.S. Pat. Nos. 6,117,425 and 6,197,325 and WO02/085422. Covalent linking of the bioactive factor to the biomaterial is a more advanced technique allowing control of the release profile of the bioactive factor from the biomaterial. Covalent cross-linking of the bioactive factor may be performed by modifying the bioactive factor through incorporation of functional groups, which are able to react with one or more of the functional groups of the precursor components or biomaterials during or after formation of the biomaterial. The incorporation of small synthetic or naturally occurring molecules, peptides and/or proteins into fibrin matrices through action of transglutaminases has been described in U.S. Pat. Nos. 6,331,422; 6,468,731 and 6,960,452 and WO 03/052091. With regard to synthetic biomaterials, thiol groups in the bioactive factor are potent groups which may react with a variety of functional groups in the synthetic precursor components or biomaterials under suitable conditions as described, for example, in WO 00/44808. The release mechanism of the bioactive factor from the biomaterial may be achieved through hydrolysis of the thioester bond thus formed.

Although the covalent incorporation can be designed such that the bioactive factor is released from the biomaterial in its wild, unmodified form, the linking mechanism of bioactive factors to synthetic precursor components or synthetic biomaterials and the resulting biomaterials described in the prior art show disadvantages. For example, the incorporation of additional cysteine/thiol groups in peptides and in particular proteins, such as growth factors, may lead to wrongly established disulfide bonds in the refolding process and as a result to inactivity of the peptide or protein. The alternative, the incorporation of amine groups instead of thiol groups can lead to unspecific and non-controllable cross-linking of the bioactive factor to the precursor components and/or biomaterials, since the reaction of amines, even to highly active functional groups of the biomaterial/precursor component, are much less specific than the reaction of thiol groups to the same functional groups.

Further, the nature of the linkage formed by reacting thiols or amines to functional groups of the precursor components and/or biomaterials may be sensitive to hydrolysis and thus the release of the bioactive factors from the biomaterial depends largely on the hydrolytic environment and is hardly controllable.

It is an object of the present invention to provide a synthetic biomaterial having incorporated therein bioactive factors which are released from the biomaterial by mechanisms other than hydrolysis.

It is a further object of the present invention to provide a mechanism for linking bioactive factors selectively to specific sides in synthetic precursor components and/or biomaterials.

In still another object of the present invention to improve the controlled release of bioactive factors from synthetic biomaterials.

### SUMMARY OF THE INVENTION

Synthetic biomaterials containing bioactive factors or modified bioactive factors that are covalently bound to the synthetic precursor components and/or biomaterials by an enzymatically degradable linkage are described herein. Further described are methods to covalently bind bioactive factors to synthetic biomaterials by means of enzymatic catalysis, the biomaterials produced therewith and the bioactive factors necessary for practicing these methods. The bioactive factors contain an amino acid sequence which can serve as a substrate domain for cross-linkable enzymes. The enzyme catalyzes the cross-linking reaction between the substrate domain of the bioactive factor and functional groups of the synthetic precursor components capable of forming the biomaterial and/or synthetic biomaterial susceptible to an enzymatically catalyzed cross-linking reaction. In a preferred embodiment, the substrate domain of the bioactive factor is selected such that the bioactive factor is cross-linkable to the synthetic precursor components capable of forming the biomaterial and/or synthetic biomaterial through the action of transglutaminases, preferably by tissue transglutaminase and even more preferably through the action of Factor XIIIa. Preferably the substrate domain of the bioactive factor comprises a transglutaminase substrate domain, even more preferably a tissue transglutaminase substrate domain, and most preferably a Factor XIIIa substrate domain.

Some bioactive factors, like Thymosin β4, inherently provide a substrate domain for cross-linkable enzymes as part of the amino acid sequence of the peptide or protein. In cases in which the primary structure of the bioactive factor does not comprise a substrate domain for cross-linking enzymes, the bioactive factor is formed synthetically, i.e. by chemical synthesis or recombinantly as a bidomain or chimeric molecule, in which the first domain comprises a substrate domain for cross-linking enzymes and the second domain comprises the bioactive factor. As generally used herein a “bidomain bioactive factor” means a bioactive factor in which an enzymatically cross-linkable substrate domain is attached to the sequence or more generally molecular structure of the bioactive factor. The covalent cross-linking of bidomain bioactive factors by enzymatic catalysis to suitable synthetic precursor components capable of forming a biomaterial and/or synthetic biomaterials is a preferred embodiment.

The functional groups of the synthetic precursor components capable of forming the biomaterial and/or synthetic biomaterial are chosen such that (i) they are cross-linkable to the substrate domain of the bioactive factor by a cross-linking enzyme, preferably by a tissue transglutaminase, and even more preferred by Factor XIIIa and and (ii) they are cross-linkable, if necessary, to the same or different precursor components to form the biomaterial. The synthetic precursor components capable of forming the biomaterial, can be linear or branched having the functional group preferably at their end termini. In a preferred embodiment the functional groups of the synthetic precursor components and/or synthetic biomaterial able to react with the enzymatically cross-linkable substrate domain of the bioactive factor are amine groups and in particular primary amine groups. In addition to the functional groups that serve as a reaction partner for the bioactive factor, there are further functional groups in the precursor component in order to form the biomaterial, preferably in-situ formation of the biomaterial. The functional groups involved in the formation of the biomaterial can be the same or different from the functional groups involved in the cross-linking of the bioactive factor. The biomaterial can be used for purposes of local drug delivery, for tissue repair and engineering of any kind of hard or soft tissue, such as repair and regeneration of injured and diseased skin, bone, tendons and cartilage.

### DETAILED DESCRIPTION OF THE INVENTION

I. Definitions

“Adhesion site or cell attachment site” as generally used herein refers to a peptide sequence to which a molecule, for example, an adhesion-promoting receptor on the surface of a cell, binds.

“Biomaterial” as generally used herein refers to a polymer, preferably a cross-linked three-dimensional polymeric network which, depending of the nature of the matrix, can be swollen with water but not dissolved in water, i.e. form a hydrogel which stays in the body for a certain period of time. Biomaterials are intended to interface with biological systems to evaluate, treat, augment, repair, regenerate or replace any tissue, organ or function of the body depending on the material either permanently or temporarily. “Natural biomaterials” as used herein refers to biomaterials that exist in nature and can be isolated therefrom or synthetically reengineered. “Synthetic biomaterials” as used herein refer to biomaterials that do not exist in nature. The terms “biomaterial”and “matrix” are used synonymously herein.

“Biocompatibility” or “biocompatible” as generally used herein refers to the ability of a material to perform with an appropriate host response in a specific application. In the broadest sense “Biocompatibility” or “biocompatible” means lack of adverse effects to the body in a way that would outweigh the benefit of the material and/or treatment to the patient.

“Bioactive factor” as generally used herein refers to a synthetic or naturally occurring molecule, nucleotide, peptide or protein which have a pharmaceutical effect on the human or animal body. The bioactive factor can be isolated from natural sources or is produced synthetically or recombinantly.

“Bidomain bioactive factor” as used herein refers to a bioactive factor in which the first domain comprises an enzymatically cross-linkable substrate domain and the second domain comprises the bioactive factor. Thus the substrate domain is not inherently part of the bioactive factor. An enzymatic degradation site can also be present between the first and the second domain and is abbreviated as “pl”. Thus, if the bidomain bioactive factor comprising a degradation site is cleaved at the degradation site, the bioactive factor is released. Cross-linkable enzymes, like tissue transglutaminases and in particular Factor XIIIa, can catalyze the formation of the covalent bond between the substrate domain of the bioactive factor and the suitable functional group of the precursor components or biomaterials.

“Biological activity” as generally used herein refers to functional events mediated by a bioactive factor of interest. In some embodiments, biological activity is measured by measuring the interactions of a polypeptide with another polypeptide. It other embodiments, biological activity is measured by assaying the effect which the protein of interest has on cell growth, differentiation, death, migration, adhesion, interactions with other proteins, enzymatic activity, protein phosphorylation or dephosphorylation, transcription, or translation.

“Conjugated unsaturated bond” as generally used herein refers to the alternation of carbon-carbon, carbon-heteroatom or heteroatom-heteroatom multiple bonds with single bonds. Such bonds can undergo addition reactions. Conjugated unsaturated bonds may undergo addition reactions for the linking of a functional group to a macromolecule, such as a synthetic polymer or a protein.

“Conjugated unsaturated group” as generally used herein refers to a molecule or a region of a molecule, which contains an alternation of carbon-carbon, carbon-heteroatom or heteroatom-heteroatom multiple bonds with single bonds, which has a multiple bond which can undergo addition reactions. Examples of conjugated unsaturated groups include, but are not limited to, vinyl sulfones, acrylates, acrylamides, quinones, and vinylpyridiniums, for example, 2- or 4-vinylpyridinium and itaconates.

“Cross-linking” as generally used herein means the formation of more than one covalent linkage within or between molecules.

“Functionalize” as generally used herein refers to modify a molecule in a manner that results in the attachment of a functional group or moiety. For example, a molecule may be functionalized by the introduction of a molecule which makes the molecule a strong nucleophile or a conjugated unsaturation. Preferably a molecule, for example PEG, is functionalized to include a thiol, amine, acrylate, or quinone group. Proteins, in particular, may also be effectively functionalized by partial or complete reduction of disulfide bonds, to create free thiols.

“Functionality” as generally used herein refers to the number of reactive sites on a molecule.

“Hard tissue” means bone, cartilage, tendon or ligament.

“Hydrogel” means a class of polymeric materials which are swollen in an aqueous medium, but which do not dissolve in water

“Multifunctional” as generally used herein refers to more than one electrophilic and/or nucleophilic functional group per molecule (i.e. monomer, oligomer or polymer).

“Polymeric network” as generally used herein means the product of a process in which substantially all of the monomers, oligomers or polymers are bound by intermolecular covalent linkages through their available functional groups to result in one large molecule, which act as the biomaterial.

“Precursor components” as generally used herein means the monomers, oligomers and/or polymers suitable for forming the biomaterial.

“Physiological” as generally used herein means conditions as they can be found in living vertebrates. In particular, physiological conditions refer to the conditions in the human body such as temperature, pH, etc. Physiological temperatures means in particular a temperature range of between 35° C. to 42° C., preferably around 37° C.

“Regenerate” as generally used herein means to grow back a portion or all of something, such as hard tissue, e.g. bone, or soft tissue, e.g. skin.

“Sensitive biological molecule” as generally used herein refers to a molecule that is found in a cell, or in a body, which may react with other molecules in its presence. Biomaterials can be made in the presence of sensitive biological materials, without adversely affecting the sensitive biological materials.

“Self selective reaction” as generally used herein means that the first precursor component of a composition reacts much faster with the second precursor component of the composition and vice versa than with other compounds present in a mixture or at the site of the reaction. As used herein, the nucleophile preferentially binds to a electrophile and an electrophile preferentially binds to a strong nucleophile, rather than to other biological compounds.

“Soft tissue” means in particular non-skeletal tissue, i.e. all tissue exclusive of bones, ligaments, tendons and cartilage, and includes spinal disc and fibrous tissue.

“Strong nucleophile” as generally used herein refers to a molecule which is capable of donating an electron pair to an electrophile in a polar-bond forming reaction. Preferably the strong nucleophile is more nucleophilic than water at physiologic pH. Examples of strong nucleophiles are thiols and amines.

“Supplemented Biomaterial” as generally used herein refers to a biomaterial having incorporated therein bioactive factors.

II. Compositions

Synthetic biomaterials having incorporated therein bioactive factors and methods for their production and use in soft and hard tissue repair, regeneration and/or remodeling, in particular for skin, bone and cartilage regeneration, are described herein. The bioactive factor is covalently cross-linked into and may be released from the synthetic biomaterial through enzymatic interaction. The synthetic biomaterials are biocompatible and biodegradable and can be formed minimally invasively in vitro or in vivo, at the site of implantation. Bioactive factors can be incorporated into the biomaterial at very specific pre-designed sites in the biomaterial such that they retain their full bioactivity once released. The bioactive factors can be releasably incorporated, using techniques that provide control over how, when and to what degree the bioactive factor is released, so that the biomaterial can be used as a controlled release vehicle. The synthetic biomaterial may further contain stabilizing materials enhancing the mechanical characteristics of the biomaterial. Examples of suitable stabilizing materials are hydroxyapatites, bone cements, calcium phosphates, calcium sulfates, etc.

A. Synthetic Biomaterials

Biomaterials for application to the human or animal body can be prepared in a variety of ways. Some biomaterials are prepared through free-radical polymerization between two or more precursor components containing unsaturated double bonds, such as described in Hem, et al., J. Biomed. Mater. Res. 39:266-276, 1998. Other biomaterials are prepared by reacting a first precursor component containing two or more nucleophilic groups, X, with at least a second precursor component containing two or more electrophilic groups, Y, which are capable of cross-linking with the nucleophilic group on the first precursor component. The reaction mechanism involved can be a nucleophilic substitution reaction, such as disclosed in U.S. Pat. No. 5,874,500, a condensation reaction and/or a Michael type addition reaction, such as described in WO 00/044808. Suitable nucleophilic groups, X, include: —NH₂, —SH, —OH, —PH₂, and —CO—NH—NH₂. Suitable electrophilic groups, Y, include: O₂N(COCH₂)₂, —CO₂H, CHO, —CHOH₂, —N═C═O, —SO₂CH═CH₂, —N(COCH), and —S—S—(C₅H₄N). A precursor component may have one or more nucleophilic groups, where the nucleophilic groups may be the same or different from each other. The second precursor component may have one or more electrophilic groups, where the electrophilic groups may be the same or different from each other. Thus a precursor component may have two or more different functional groups.

1. Michael Type Addition Reaction

The 1,4 addition reaction of a nucleophilic group on a conjugate unsaturated system is referred to as a Michael type addition reaction. The preferred cross-linking mechanism for the formation of biomaterials is through a Michael type addition reaction. A Michael type addition reaction allows for in situ cross-linking at the site of need in the body of at least a first and a second precursor component under physiological conditions in a self-selective manner, even in the presence of sensitive biological materials. Thus the first precursor component reacts much faster with a second precursor component than with other components in the sensitive biological environment, and the second precursor component reacts much faster with the first precursor component than with other components in the sensitive biological environment present in the body. When one of the precursor components has a functionality of at least two, and at least one of the other precursor components has a functionality of greater than two, the system will self-selectively react to form a cross-linked three dimensional biomaterial.

In the Michael type addition reaction, the addition mechanism can be purely polar, or can proceed through a radical-like intermediate state(s). Lewis acids or bases, or appropriately designed hydrogen bonding species, can act as catalysts. The term “conjugation” can refer both to alternation of carbon-carbon, carbon-heteroatom or heteroatom-heteroatom multiple bonds with single bonds, or to the linking of a functional group to a macromolecule, such as a synthetic polymer or a protein. Double bonds spaced by a CH or CH₂ unit are referred to as “homoconjugated double bonds”. Michael type addition to conjugated unsaturated groups to form biomaterials can take place in substantially quantitative yields at physiological temperatures, in particular at body temperature, but also at lower and higher temperatures than body temperature. These reactions take place in mild conditions with a wide variety of nucleophilic groups. The biomaterial formation kinetics and the mechanical and transport properties of the biomaterial are tailored to the needs of the application.

a. Nucleophilic Groups for Carrying Out a Michael Type Addition Reaction

The nucleophilic groups of a precursor component (either the first or second precursoru component) useful for carrying out the Michael type addition reaction are able to react with conjugated unsaturated groups. The nucleophilic groups are selected such that they are reactive towards conjugated unsaturated groups under conditions as they are present in the human or animal body. The reactivity of the nucleophilic groups depends on the identity of the unsaturated group, but the identity of the unsaturated group is first limited by its reaction with water at physiologic pH. Thus, the useful nucleophilic groups are more nucleophilic than water at physiologic pH. Preferred nucleophilic groups are ones that are commonly found in biological systems, for reasons of toxicology, but ones that are not commonly found free in biological systems outside of cells. Thus, preferred nucleophilic groups are thiols and amines, and most preferred are thiols.

Thiols are present in biological systems outside of cells in paired form, as disulfide linkages. When the highest degree of self-selectivity is desired (e.g. when the cross-linking reaction is conducted in the presence of tissue and chemical modification of that tissue is not desirable), then a thiol will represent the strong nucleophilic group of choice.

There are other situations, however, when the highest level of self-selectivity may not be necessary. In these cases, an amine may serve as an adequate nucleophilic group. Here, particular attention is paid to the pH, in that the deprotonated amine is a much stronger nucleophile than the protonated amine. Thus, for example, the alpha amine on a typical amino acid (pK as low as 8.8 for asparagine, average of 9.0 for all 20 common amino acids except proline) has a much lower pK than the side chain epsilon amine of lysine (pK 10.80). As such, if particular attention is paid to the pK of an amine used as the strong nucleophile, substantial self-selectivity can be obtained. By selecting an amine with a low pK, and then formulating the final precursor such that the pH were near that pK, one could favor reaction of the unsaturation with the amine provided, rather than other amines present in the system. In cases where no self-selectivity is desired, one need pay less attention to the pK of the amine used as the nucleophile. However to obtain reaction rates that are acceptably fast, one must adjust the pH of the final precursor solution such that an adequate number of these amines are deprotonated.

In summary, the usefulness of particular nucleophilic groups depends upon the situation envisioned and the amount of self-selectivity desired. Thiols are generally the \ preferred strong nucleophile of this invention, due to the pH in the precursor mixture and to obtain maximal self-selectivity, but there are situations in which amines will also serve as useful strong nucleophilic groups.

The concept of nucleophilic group is extended herein, so that the term is sometimes used to include not only the functional groups themselves (e.g., thiol or amine), but also molecules which contain the functional group.

The nucleophilic groups may be contained in molecules, typically one of the precursor components, with great flexibility in overall structure. For example, a difunctional nucleophile could be presented in the form of X-P-X, where P refers to a precursor component, i.e. the monomer, oligomer or polymer, and X refers to the nucleophilic group. Likewise, a branched polymer, P, could be derivatized with a number of nucleophilic groups to create P-(X)ᵢ. X need not be displayed at the chain termini of P, for example, a repeating structure could be envisioned: (P-X)ᵢ. Not all of the P or the X in such a structure need to be identical.

b. Electrophilic Groups for the Michael Type Addition Reaction

The electrophilic groups of a precursor component (either the first or second precursor component) useful for carrying out a Michael type addition reaction are preferably conjugated unsaturated groups.

The structures of a presurcor component, P, and the conjugated unsaturated groups may be similar to those described in detail above with respect to the nucelophilic groups. It is only necessary that the precursor component contains at least two such conjugated unsaturated groups (i.e. greater than or equal to two such conjugated unsaturated groups).

It is possible to perform nucleophilic addition reactions, in particular Michael addition reactions, on a wide variety of conjugated unsaturated compounds. In the structures shown below, a precursor component can be monomeric, oligomeric or polymeric structure and is indicated as P. Various preferred possibilities for the specific identity of P are discussed further herein. P can be coupled to reactive conjugated unsaturated groups in structures such as those numbered 1 to 20 and listed in Table 1.

TABLE 1
Selected Conjugated Unsaturated Groups

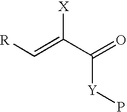

1a

X = H, CH₃, CN, COOW
R = H, W, Phenyl- (Ph)
Y = NH, O, 1,4-Ph
W = Cl—C5 aliphatic chain

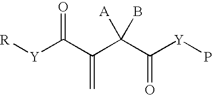

1b

A, B = H, alkyl
R = H, alkyl
Y = O, NH, 1,4-Ph

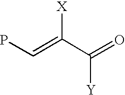

2

X = CN, COOW
Y = OW, Ph
W = Cl—C5 aliphatic chain

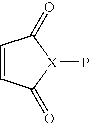

3

X = N, CH

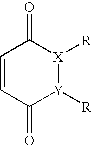

4

| | |
|---|---|
| A | X = CH |
| | Y = CH |
| | R = H, W-P(W = NH, O, nihil) |
| B | X = N |
| | Y = N |
| | R = H, P |
| C | X—Y = C═C |
| | R = W-P (W = NH, O, nihil) |

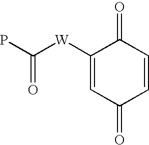

5

W = NH, O, nihil

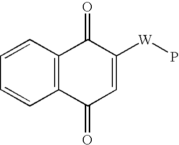

6

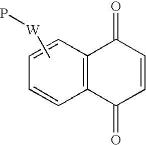

7

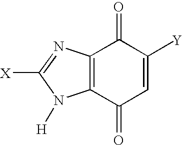

8

| | |
|---|---|
| X,Y = | H, P |
| | P, P |
| | P, H |
| | P, aliphatic chain |

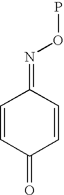

9

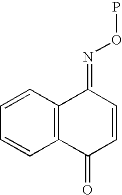

10

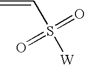

11

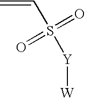

13

Y = O, NH
X = alkali or
alkali earth
metal ion, P
W = P, 1,4-Ph-P

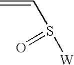

12

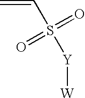

14

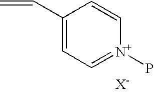

15

X = halogen, sulphonate

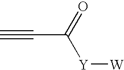

16

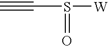

17

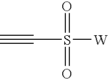

18

W = P

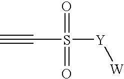

19

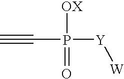

20

Y = O, NH
X = alkali or alkali
earth
metal ion, P
W = P, 1,4-Ph-P

Reactive double bonds can be conjugated to one or more carbonyl groups in a linear ketone, ester or amide structure (1a, 1b, 2) or to two in a ring system, as in a maleic or paraquinoid derivative (3, 4, 5, 6, 7, 8, 9, 10). In the latter case, the ring can be fused to give a naphthoquinone (6, 7, 10) or a 4,7-benzimidazoledione (8) and the carbonyl groups can be converted to an oxime (9, 10). The double bond can be conjugated to a heteroatom-heteroatom double bond, such as a sulfone (11), a sulfoxide (12), a sulfonate or a sulfonamide (13), a phosphonate or phosphonamide (14). Finally, the double bond can be conjugated to an electron-poor aromatic system, such as a 4-vinylpirydinium ion (15). Triple bonds can be used in conjugation with carbonyl or heteroatom-based multiple bonds (16, 17, 18, 19, 20).

Structures such as 1a, 1b and 2 are based on the conjugation of a carbon-carbon double bond with one or two electron-withdrawing groups. One of them is always a carbonyl, increasing the reactivity passing from an amide, to an ester, and then to a phenone structure. The nucleophilic addition is easier upon decreasing the steric hindrance, or increasing the electron-withdrawing power in the alpha-position: CH₃<H<COOW<CN.

The higher reactivity obtained by using the last two structures can be modulated by varying the bulkiness of the substituents in the beta-position, where the nucleophilic attack takes place; the reactivity decreases in the order P<W<Ph<H. So, the position of P too can be used to tune the reactivity towards nucleophilic groups. This family includes some compounds for which a great deal is known about their toxicology and use in medicine. For example, water-soluble polymers with acrylates and methacrylates on their termini are polymerized (by free radical mechanisms) in vivo. Thus, acrylate and methacrylate-containing polymers have been seen in the body before in clinical products, but for use with a dramatically different chemical reaction scheme.

The structures 3-10 exhibit very high reactivity towards nucleophilic groups, due both to the cis configuration of the double bond and the presence of two electron-withdrawing groups. Unsaturated ketones react faster than amides or imides, due to the stronger electronegativity of these carbonyl groups. Thus, cyclopentendione derivatives react faster than maleimidic ones (3), and para-quinones react faster than maleic hydrazides (4) and also faster than cyclohexanones, due to more extended conjugation. The highest reactivity is shown by naphthoquinones (7). P can be placed in positions where it does not reduce the reactivity of the unsaturated group, that is in the opposite part of the ring (3, 5), on another ring (7, 8) or O-linked through a para-quinone mono-oxime (9, 10). P can be also linked to the reactive double bond (6, 8), if the nucleophilic addition rate is to be decreased.

The activation of double bonds to nucleophilic addition can also be obtained by using heteroatoms-based electron-withdrawing groups. In fact, heteroatom-containing analogues of ketones (11, 12), esters and amides (13, 14) provide a similar electronic behavior. The reactivity towards nucleophilic addition increases with electronegativity of the group, that is in the order 11>12>13>14, and is enhanced by the linkage with an aromatic ring. A strong activation of double bonds can also be obtained, using electron-withdrawing groups based on aromatic rings. Any aromatic structure containing a pyridinium-like cation (e.g., derivatives of quinoline, imidazole, pyrazine, pyrimidine, pyridazine, and similar Sp₂-nitrogen containing compounds) strongly polarizes the double bond and makes possible quick Michael type additions.

Carbon-carbon triple bonds, conjugated with carbon- or heteroatom-based electron-withdrawing groups, can easily react with sulphur nucleophiles, to give products from simple and double addition. The reactivity is influenced by the substituents, as for the double bond-containing analogous compounds.

The formation of ordered aggregates (liposomes, micelles) or the simple phase separation in water environment increases the local concentration of unsaturated groups and so the reaction rate. In this case, the latter depends also on the partition coefficient of the nucleophilic groups, which increases for molecules with enhanced lipophilic character.

B. Precursor Components, P

The first and second precursor components can be monomeric, oligomeric or polymeric and are abbreviated herein as “P”. Suitable precursor components include proteins, peptides, polyoxyalkylenes, poly(vinyl alcohol), poly(ethylene-co-vinyl alcohol), poly(acrylic acid), poly(ethylene-co-acrylic acid), poly(ethyloxazoline), poly(vinyl pyrrolidone), poly(ethylene-co-vinyl pyrrolidone), poly(maleic acid), poly(ethylene-co-maleic acid), poly(acrylamide), or poly(ethylene oxide)-co-poly(propylene oxide) block copolymers. Particularly preferred for the first and second precursor component is polyethylene glycol (PEG). In another preferred embodiment the second precursor component is a synthetic peptide.

Functionalized PEG has been shown to combine particularly favourable properties in the formation of synthetic biomaterials. Its high hydrophilicity and low degradability by mammalian enzymes and low toxicity make PEG particularly useful for application in the body. One can readily purchase or synthesize linear (meaning with two ends) or branched (meaning more than two ends) PEGs and then functionalize the PEG end groups according to the reaction mechanisms of choice.

In one preferred embodiment, the first component is a trifunctional three arm 15 kDa polymer, i.e. each arm having a molecular weight of 5 kDa, and the second precursor component, wherein the second precurspor component is a bifunctional linear molecule of a molecular weight in the range of between 0.5 to 1.5 kDa, even more preferably around 1 kDa. Preferably the first and the second precursor components are polyethylene glycol molecules.

In another preferred embodiment, the first precursor component is a four arm 15 kDa to 20 kDa polymer having functional groups at the terminus of each arm and the second precursor component is a bifunctional linear molecule with a molecular weight in the range of between 1 to 4 kDa, preferably around 3 to 4 kDa, and most preferrably 3.4 kDa. Preferably the first precursor component comprises conjugated unsaturated groups or bonds, preferably an acrylate or a vinylsulfone, and most preferably an acrylate, and the second precursor component comprises a nucleophilic group, preferably a thiol or amine groups.

Preferably the first precursor component is a polyethylene glycol, and the second precursor component is a peptide or also a polyethylene glycol. In the most preferred embodiment, both precursor components are polyethylene glycol molecules. One preferred embodiment is a biomaterial made of the combination of a four-arm 15 kD PEG acrylate and a 3.4 kD linear PEG thiol.

C. Cell Attachment Sites

In a further preferred embodiment, peptide sites for cell adhesion are incorporated into the biomaterial. The cell attachment sites are peptides that bind to adhesion-promoting receptors on the surfaces of cells. Examples of adhesion sites include, but are not limited to, RGD sequence and YIGSR (SEQ ID NO: 1). Particularly preferred are the RGD sequence from fibronectin, the YIGSR (SEQ ID NO: 1) sequence from laminin. The incorporation can be done, for example, simply by mixing a cysteine-containing cell attachment peptide with the precursor component including a conjugated unsaturated group, such as PEG acrylate, PEG acrylamide or PEG vinylsulfone, a few minutes before mixing with the remainder of the precursor component including the nucleophilic group, such as thiol-containing precursor component. If the cell attachment site does not include a cysteine, it can be chemically synthesized to include one. During this first step, the adhesion-promoting peptide will become incorporated into one end of the precursor multiply functionalized with a conjugated unsaturation; when the remaining multithiol is added to the system, the biomaterial will form.

D. Bioactive Factors

A wide range of bioactive factors can be incorporated into the synthetic biomaterials. Suitable bioactive factors include nucleotides, peptide or proteins able to induce and support healing, repair and regeneration of soft and hard tissue, in particular skin, bone and cartilage. Preferred bioactive factors include parathyroid hormones (PTHs), platelet-derived growth factors (PDGFs), Transforming growth factor betas (TGF βs), bone morphogenetic proteins (BMPs), vascular endothelial growth factor (VEGFs), Insulin-like growth factors (IGFs), Fibroblast Growth Factors (FGFs), and variants having the same effect in the human or animal body. Most preferred bioactive factors include PDGF AB, PTH₁₋₃₄, BMP2, BMP 7, TGF β1, TGF β3, VEGF 121, and VEGF 110. Other suitable bioactive factors include antibiotics, anti-cancer drugs, pain-reducing drugs, antiproliferating agents, etc.

In one embodiment, the bioactive factor is a bidomain bioactive factor, where the first domain comprises an enzymatically cross-linkable substrate domain and the second domain comprises the bioactive factor. Optionally, the bidomain bioactive factor contains an enzymatic degradation site (abbreviated as “pl”) between the first and the second domain. This allows for the controlled relase of the bioactive factor. The most preferred bidomain bioactive factors for incorporation in a synthetic precursor component or synthetic biomaterial is the combination of a Factor XIIIa substrate domain (“TG-sequence”) including a plasmin degradable site and one of the preferred or most preferred bioactive factors listed above. However it is understood that any other bioactive factor, like antibiotics, anti-cancer drugs, pain-reducing drugs, etc, can be included in the bidomain bioactive factor and incorporated into the biomaterial.

1. Enzymatically Cross-Linkable Substrate Domains of the Bioactive Factor

The bioactive factor and, in particular, the bidomain bioactive factor can be cross-linked to appropriate functional groups of the precursor components and/or biomaterials through the cross-linkable substrate domain of the bioactive factors and/or bidomain bioactive factor. The substrate domain is a domain for an enzyme, preferably a substrate domain for a transglutaminase, more preferably for a tissue transglutaminase, (“TR-domain”), and even more preferably for Factor XIIIa.

Mammalian transglutaminases are encoded by a family of structurally and functionally related genes. Nine transglutaminase genes have been identified, eight of which encode active enzymes. The transglutaminase enzyme family includes: (a) the intracellular transglutaminases 1, 3 and 5 isoforms, which are mostly expressed in epithelial tissue; (b) transglutaminase 2 which is expressed in various tissue types and occurs in an intracellular and an extracellular form; (c) transglutaminase 4, which is expressed in prostate gland; (d) Factor XIIIa (abbreviated “FXIIIa”) which is expressed in blood; (e) transglutaminase 6 and 7, whose tissue distribution is unknown and (f) band 4.2, which is a component protein of the membrane that has lost its enzymatic activity, and serves to maintain erythrocyte membrane integrity. In most instances, transglutaminases catalyse acyl-transfer reactions between the gamma-carboxamide group of protein bound glutaminyl residues and the epsilon-amino group of lysine residues, resulting in the formation of N-epsilon-(gamma-glutamyl)lysine isopeptide side chains bridges. The amino acid sequence of the enzymatically cross-linkable substrate domain can be designed to further contain a cleavage site, the bioactive factor can be released with little or no modification to the primary structure, which may result in higher activity of the bioactive factor. If the cleavage site is enzymatically degradable, the release of the bioactive factor is controlled by cell specific processes, such as localized proteolysis. Conservation of bioactive factors, in particular in the case of growth factors and their bioavailability, are distinct advantages of exploiting cell specific proteolytic activity over the use of diffusion controlled release devices which characteristically result in the loss of a significant amount of bioactive factor in an initial burst release. These degradable sites allow for the engineering of more specific release of bioactive factors from synthetic biomaterials. Transglutaminase substrate domains and their amino acid sequences are listed in Table 2.

TABLE 2
Transglutaminase substrate domains

| | |
|---|---|
| SEQ ID NO: 2 | GAKDV |
| | A peptide that mimics the lysine coupling site in |
| | the chain of fibrinogen |
| SEQ ID NO: 3 | KKKK |
| | A peptide with a polylysine at a random coupling |
| | site |
| SEQ ID NO: 4 | NQEQVSPL |
| | A peptide that mimics the cross-linking site in |
| | α2-plasmin inhibitor (abbreviated TG) |
| SEQ ID NO: 5 | YRGDTIGEGQQHHLGG |
| | A peptide with glutamine at the transglutaminase |
| | coupling site in the chain of fibrinogen |

As generally used herein, the tissue transglutaminase substrate domain is abbreviated “TR-domain”, and the bioactive factor modified by a transglutaminase substrate domain is abbreviated “TR-bioactive factor”. The TR-domain may include GAKDV(SEQ ID NO: 2) and KKKK (SEQ ID NO: 3). The production of the bidomain bioactive factor is dependent on the nature of the bioactive factor; it can be performed by chemical synthesis or recombinant technologies. For example TR-PTH can be produced by chemical synthesis, whereas TR-growth factors, like TR-PDGF or TR-BMP, TR-IGF are produced by bacterial or mammalian recombinant expression systems with subsequent refolding and purification steps.

The most preferred Factor XIIIa substrate domain has an amino acid sequence of NQEQVSPL (SEQ ID NO: 4) and is herein referred to as “TG” and TG-bioactive factor. Other proteins that transglutaminase recognizes, such as fibronectin, could be coupled to the bioactive factor.

a. Degradation Sites in the Enzymatically Cross-Linkable Substrate Domain of the Bioactive Factor

The cross-linkable substrate domain of the bidomain bioactive factor preferably includes an enzymatically degradable amino acid sequence, so that the bioactive factor can be cleaved from the biomaterial by enzymes in substantially the unmodified form. In particular a plasmin degradable sequence is attached as a linker between the bioactive factor and the enzymatically cross-linkable substrate domain. The sequence GYKNR (SEQ ID NO: 6) between the first domain and the second domain of the bidomain bioactive factor makes the linkage plasmin degradable. Thus most preferred bidomain bioactive factors are TGplPDGF AB, TG-plPTH₁₋₃₄, TGplBMP2, TGplTGFβ3, TGplVEGF 121, and TGplVEGF 110.

Degradation based on enzymatic activity allows for the release of the bioactive factor to be controlled by a cellular process rather than by diffusion of the factor through the biomaterial. The degradable site or linkage is cleaved by enzymes released from cells while they invade, degrade and stay within the matrix. This allows bioactive factors to be released at different rates within the same biomaterial depending on the location of cells within the material. This also reduces the amount of total bioactive factor needed, since the release is over time and controlled by cellular processes. Conservation of bioactive factors and its bioavailability are distinct advantages of exploiting cell specific proteolytic activity over the use of diffusion controlled release devices. In one possible explanation for the strong healing of a bone defect with TGplPTH₁₋₃₄ or TGplBMP2 covalently bound to a synthetic biomaterial, it is deemed important that the PTH or BMP2 is administered locally over an extended period of time and in the case of PTH not just as a single pulsed dose. The same holds true for TGplPDGF AB cross-linked to a synthetic biomaterial. Finally, the therapeutic effects of the bioactive factors are localized to the defect region and are subsequently magnified.

Enzymes that can be used for proteolytic degradation are numerous. Proteolytically degradable sites could include substrates for collagenase, plasmin, elastase, stromelysin, or plasminogen activators. Exemplary substrates are listed below in Table 3. N1-N5 denote amino acids 1-5 positions toward the amino terminus of the protein from the site were proteolysis occurs. N1′-N4′ denote amino acids 1-4 positions toward the carboxy terminus of the protein from the site where proteolysis occurs.

TABLE 3
Sample Substrate Sequences for Protease

| | | | | | | | | | | SEQ ID |
|---|---|---|---|---|---|---|---|---|---|---|
| Protease | N | N4 | N3 | N2 | N1 | N1′ | N2′ | N3′ | N4′ | NO: |
| Plasmin¹ | | | L | I | K | M | K | P | | SEQ ID |
| | | | | | | | | | | NO: 6 |
| Plasmin¹ | | | N | F | K | S | Q | L | | SEQ ID |
| | | | | | | | | | | NO: 8 |
| Stromelysin² | Ac | G | P | L | A | L | T | A | L | SEQ ID |
| | | | | | | | | | | NO: 9 |
| Stromelysin² | | Ac | P | F | E | L | R | A | NH₂ | SEQ ID |
| | | | | | | | | | | NO: 10 |
| Elastase³ | | | Z− | A | A | F | A | NH₂ | | SEQ ID |
| | | | | | | | | | | NO: 11 |
| Collagenase⁴ | | G | P | L | G | I | A | G | P | SEQ ID |
| | | | | | | | | | | NO: 12 |
| t-PA⁵ | P | H | Y | G | R | S | G | G | | SEQ ID |
| | | | | | | | | | | NO: 14 |
| u-PA⁵ | P | G | S | G | R | S | A | S | G | SEQ ID |
| | | | | | | | | | | NO: 15 |

References:
¹Takagi and Doolittle, (1975) Biochem. 14: 5149-5156.
²Smith et al., (1995). J. Biol. Chem. 270: 6440-6449.
³Besson et al., (1996) Analytical Biochemistry 237: 216-223.
⁴Netzel-Arnett et al., (1991) J. Biol. Chem. 266: 6747-6755.
⁵Coombs et al., 1998. J. Biol. Chem. 273: 4323-4328.

III. Methods of Incorporating the Bioactive Factors into the Biomaterials and/or Precursor Component(s)

There are several ways of linking the bioactive factor or bidomain bioactive factor to the synthetic precursor component capable of forming the biomaterial or the synthetic biomaterial.

A. Amine-Modified Precursor Component

1. First Method: Amine-Modified Precursor Component

Generally in a first step an amine or multiamine modified precursor component is formed. This precursor component can be either linear or branched as described hereinbefore. For example, an amino terminated precursor component, like linear or branched PEG amine, polyamines, polyimides, polyimines, may be provided, e.g Nektar Therapeutics, US or formed by known synthesis.

2. Second Method: Formation of Amine-modified Precursor Component Using a Bidomain Linker

In another method a precursor component comprising conjugated unsaturated groups is provided, preferably the conjugated unsaturated groups are located at the end-terminus of the precursor component. Those molecules have been described hereinbefore.

In the next step the precursor component reacts with a multifunctional linker molecule, which comprises at least one thiol as well as at least one amine group, the amine group is preferably a primary amine group. The preferred linker molecule is generally expressed as HS—(X)ₙ—NH₂ or HS—(Xᵢ)ₙ—NH₂. X can be any suitable group or atom as long as it does not hinder the reactions of —HS and —NH₂, and it can be branched or linear. HS—(X)ₙ—NH₂ or HS—(Xᵢ)ₙ—NH₂ can be selected from a variety of molecules like, cysteine containing natural peptides, hormones or proteins, any synthetic peptide comprising cysteine like CRGD (SEQ ID NO: 15). Further mercapto-amines, such as for example mercaptoethylamine, are suitable. Preferably X is a methylene group (—CH2—); n is preferably selected from higher than 2.

In a preferred embodiment the linker molecule is selected from the group consisting of synthetic or natural peptides of the formula (CXKX), like CGKG (SEQ ID NO: 16). The amino acid lysine K participates in the cross-linking reaction performed by the Factor XIIIa, C provides the thiol group to react in a Michael type addition reaction with a conjugated unsaturated group of a precursor componentt and X can be any molecule or atom which does not aversively affect the cross-linking reaction. In another preferred embodiment, the linker molecule has the amino acid sequence CRGD (SEQ ID NO: 15), where the functionality of RGD which acts as a cell attachment site is combined with the thiol and amine functionality. In another preferred embodiment X is a methylene group and n is greater than 2. Mercaptoethylamine has shown good performance.

The thiol group of the linker reacts in a Michael addition reaction with the conjugated unsaturated group at the end-terminus of the precursor component, which leads to a free primary amino group at the terminus of the resulting amine precursor component.

B. Enzymatically Catalyzed Cross-Linking Reaction Between the Bioactive Factor and the Amine Precursor Component

Once the amine precursor component or multi-amine precursor component is formed, it serves in a next step as the reaction partner in the enzymatically catalyzed cross-linking reaction between the bioactive factor or bidomain bioactive factor and the amine (or multi-amine) precursor component. Preferably, the cross-linking reaction is catalyzed by transglutaminase. In case of a TG-bioactive factor, the TG-bioactive factor is mixed with the amine precursor component in the presence of calcium and activated Factor XIIIa. Under physiological conditions, Factor XIIIa proceeds to link the TG-bioactive factor to the amine group of the amine precursor component, creating a covalent bond between the bioactive factor and the amine precursor component.

For example, a polyethylene glycol modified bioactive factor may be formed by (a) providing a polyethylene glycol molecule comprising at least one amine group; (b) providing a bioactive factor or bidomain bioactive factor comprising a substrate domain for a cross-linkable enzyme; (c) providing an enzyme capable of catalyzing a cross-linking reaction between the substrate domain of the bioactive factor or bidomain bioactive factor and the amine group; and (d) cross-linking the bioactive factor or bidomain bioactive factor to the amine group on the polyethylene glycol molecule.

C. Reaction Between Bioactive Factor-Precursor Component and Precursor Component Comprising Strong Nucleophilic Groups

After the formation of the bioactive factor-precursor component in the second method, this component reacts in a last step with at least a second precursor component comprising strong nucleophilic groups. The strong nucleophilic groups of the second precursor component will react with conjugated unsaturated groups of the bioactive factor-precursor component (those which were not consummated by the reaction with the bioactive factor) in a Michael addition reaction, thus forming the synthetic biomaterial supplemented with bioactive factors. In case of the first method a first precursor component containing conjugated unsaturated groups are added and the free amine groups of the precursor component (those which were not consummated by the reaction with the bioactive factor) react with the conjugated unsaturated group of the other precursor component. Preferably the ratio of the equivalent weight of the functional groups of the first and second precursor molecule is between 0.9 and 1.1 without taking into consideration the reaction with the bidomain bioactive factor or bioactive factor. The concentration of the first and second precursor component is adjusted depending on the concentration of the bioactive factor employed, in order to keep the ratio of the equivalent weight of the functional groups in the preferred range.

For example, a synthetic biomaterial comprising bioactive factors or bidomain bioactive factors cross-linked to the biomaterial, where the bioactive factors or bidomain bioactive factors comprise a substrate domain for a cross-linkable enzyme, can be formed by (a) providing a first precursor component comprising conjugated unsaturated groups, (b) providing a linker molecule comprising at least one thiol group and at least one amine group, (c) reacting a portion (i.e. not all) of the conjugated unsaturated groups with the thiol group to form an amine-modified precursor component; (d) providing an enzyme (e.g. transglutaminase) capable of catalyzing a cross-linking reaction between the substrate domain of the bioactive factors or bidomain bioactive factors and the amine group of the amine-modified precursor component; (e) reacting the amine group of the amine-modified precursor component with the substrate domain of the bioactive factors or bidomain bioactive factors to form a bioactive factor-precursor component; (f) providing a second precursor component comprising strong nucleophilic groups, and (g) reacting in a Michael addition reaction the strong nucleophilic groups of the second precursor component with the conjugated unsaturated groups of the bioactive factor-precursor component to form a biomaterial.

IV. Methods of Applying and Using the Supplemented Biomaterials

If the formation of the biomaterial is not easily reversible, such as in the case of thermoreversible biomaterials, the first and second precursor components should not be combined or come into contact with each other under conditions that allow polymerization of the precursor components prior to time that the formation of the biomaterial is desired. This is generally achieved by a system comprising at least a first and a second precursor component separated from each other. The bioactive factor or bidomain bioactive factor and/or a bifunctional linker molecule are either stored separately from the precursor components or, under appropriate conditions, are mixed and stored with one of the precursor components. The first precursor component, the second precursor component, the linker molecule and/or the bioactive factor or bidomain bioactive factor are preferably stored under exclusion of oxygen and light and at low temperatures, e.g. around +4° C., to avoid decomposition of the functional groups prior to use.

In one embodiment the enzyme, the bidomain bioactive factor and/or the linker molecule are stored together. At the time of application, they are dissolved and mixed with the dissolved precursor component, which is reactive towards an enzymatic cross-linking with the bioactive factor or bidomain bioactive factor. After the cross-linking of the bioactive factor or bidomain bioactive factor to the precursor component is completed, the bioactive factor-precursor component is mixed with the second precursor component to form the biomaterial.

The biomaterials may be used for localized or systemic delivery of the bioactive factors, for tissue repair and regeneration and in particular for regeneration of soft and hard tissue, such as skin, bone, tendons and cartilage.

Although the scope of the present invention is the formation of in-situ forming synthetic biomaterials having covalently incorporated bioactive factors, it is to be understood that that the enzymatic crosslinking reaction of bidomain bioactive factors or bioactive factors to a synthetic precursor molecule can be used, or example, to pegylate the bioactive factor for systemic application to the body.

The present invention will be further understood by reference to the following non-limiting examples.

### EXAMPLES

Materials and Methods

Table 4 provides the descriptions and abbreviations for the materials used in Examples 1 and 2.

TABLE 4
Materials and abbreviations

| | | Molecular | | Conc./ |
|---|---|---|---|---|
| Abbreviation | Product | weight | Manufacturer | Buffer |
| FXIIIa | Factor XIIIa | 312000 | Baxter | 173 U/ml |
| TG-plPDGF | TGplPDGF.AB | 34000 | Self | 2.8 mg/ml |
| TG-plPTH | TGplPTH₁₋₃₄ | 5575 | Bachem; CH | — |
| TG-plPTH- | Dansyl-TGplPTH₁₋₃₄ | 5808 | Bachem, CH | — |
| dansyl | | | | |
| Thrombin | Thrombin | 72000 | Baxter, A | 500 U/mg |
| PEG-Acr | Poly(ethylene glycol) | 14861 | Nektar; US | 100% |
| | tetraacrylate, 4 arm, | | | pure |
| | endfunctionalized | | | |
| PEG-thiol | Poly(ethylene glycol) | 3391 | Nektar | 75% |
| | dithiol | | | Pure |
| 8-arm PEG- | 8-arm poly(ethylene | 54000 | Self (Lutolf | |
| VS | glycol) vinylsulfone, | | and Hubbell, | |
| | synthesized from 8-arm | | Biomacromolecules, | |
| | PEG-OH (40 kDa, | | 4 (3), 713-722, 2003) | |
| | Nektar Therapeutics) | | | |
| DTNB | 5,5′-Diothio-bis-(2- | 396.3 | Sigma | |
| | nitrobenzoic acid), | | | |
| | Ellman's Reagent | | | |
| MEA | Mercaptoethylamine | 77.15 | Sigma | |
| PepI | Ac-FKGG-GPQGIWGQ- | 1717.9 | NeoMPS, FR | >90% |
| | ERCG (SEQ ID NO: 17) | | | by |
| | | | | HPLC |
| PepII | Ac-FKGGERCG (SEQ | 895 | NeoMPS, FR | 98.4% |
| | ID NO: 18) | | | by HPLC |
| TEA | Triethanolamine | 149.19 | Sigma | |

### Example 1

A bifunctional peptide linker molecule, Pep I, containing a Lys and Cys, (with a primary structure of Ac-FKGG-GPQGIWGQ-ERCG (SEQ ID NO: 17); where the sequence in the middle represents a degradable sequence), was conjugated to the vinylsulfone end-groups of an 8-arm end-functionalized polyethylene glycol (PEG)-macromer to form a precursor component. This peptide linker-modified precursor PEG component served as the amine component for the subsequent cross-linking of TG-plPTH and TG-pl-PDGF (TG sequence: NQEQVSPL; SEQ ID NO: 4) to form PEGylated bioactive factors.

1. Coupling of PepI to 8arm-PEG-VS via Michael-type addition. PepI was added to 8arm-PEG-VS in 1.2-fold molar excess over vinylsulfone groups in 0.3 M triethanolamine (pH 8.0) at 37° C. for 2 hours. The reaction solution was subsequently dialysed (Slide-A-Lyzer® 7K, MWCO: 7000, PIERCE, Rockford, Ill.) against ultrapure water for three days at 4° C. After dialysis the product (termed herein 8PEG-PepI) was lyophilized to obtain a white powder.

2. Factor XIIIa-catalyzed coupling of TG-plPTH and TG-plPDGF into 8PEG-VS-PepI

a) Activation of FXIIIa by thrombin. 100 μl of Factor XIIIa (170-200 U/ml) was activated with 50 μl of thrombin (20 U/ml) for 30 minutes at 37° C. Small aliquots of FXIIIa were stored at −20° C. for further use.

b) Conjugation of TG-plPTH and TG-plPDGF to 8PEG-VS-PepI. Generally, the following, previously optimized conditions were used for FXIIIa-mediated PEGylation: 4 μl of TG-plPTH (0.8 mg/ml, dissolved in PBS, pH 7.4) or TG-plPDGF (0.73 mg/ml, dissolved in PBS, pH 7.4), respectively, were added to 10.8 μl of 8PEG-VS-PepI solution (0.37 mg/ml corresponding to a ca. 7-fold molar excess of Lys donor over Gln acceptor in case of TG-plPTH; in 50 mM Tris, 50 mM CaCl₂, pH 7.6).

In a second step, 0.7 μl of activated FXIIIa (10 U/ml during reaction) were added to the above solutions.

The final solution was vortexed and reacted at room temperature for 10, 30 and 60 minutes. Reactions were stopped by immersion of the samples in liquid nitrogen and storing at −20° C. Directly after the reaction, the samples were resolved on NuPAGE™ 12% (in MES, for PTH) and 4-12% (in MES, PDGF) Bis-Tris Gels SDS-PAGE gels (Invitrogen) and Silver stained (Silver Stain Plus, BIO-RAD) according to the manufacturer's protocol.

Results and Discussions

Factor XIIIa Catalyzed Conjugation of TG-PTH to PEG-VS-PepI

SDS-PAGE with silver staining demonstrated that FXIIIa can catalyze the reaction between the glutamine acceptor peptide at the N-terminus of TG and the lysine donor peptide conjugated to PEG, thus yielding PEG-modified TG-plPTH. In the SDS-Page it can be observed that the band for TGplPTH at 4.5 kDa disappears and bands representing the reaction product appear around 49 kDa, 62 kDa and 85 kDa. This change is in comparison to TGplPTH run alone, which shows a band at only 4.5 kDa. When one of the three components required for the cross-linking reaction (TGplPTH, modified PEG and Factor XIIIa) is missing, the band at 4.5 kDa does not change. It can be seen that the TGplPTH reacts quickly and specifically with the modified PEG through the action of Factor XIIIa.

The reaction is due to the presence of FXIII, as 8PEG-VS-PepI alone does not seem to affect TG-PTH.

From a comparison of the TG-plPTH band (below the 6 kDa marker) and the same band after PEG conjugation it appears that most (estimated >90%) of the PTH had reacted. Moreover, due to the intense staining of 8PEG-VS-PepI (which showed a rather broad molecular weight distribution with main bands around 49, 62 and several bands between 62 and 98 kDa) and FXIIIa, the reaction product (PEGylated PTH, with a theoretical molecular weight around 54 kDa) is difficult to identify. Nevertheless, it seems that the bands just under 49 kDa, 62 kDa and 98 kDa correspond to PEG-conjugated PTH.

The polyacrylamide gel showed that when TGplPTH was allowed to react in the presence of activated Factor XIIIa with a PEG, end terminated with vinylsulfone, which was pre-reacted with a lysine substrate to form the tissue transglutaminase substrate domain, the band for TGplPTH at 4.5 kDa disappeared and bands representing the reaction product appeared around 49 kDa, 62 kDa and 85 kDa. This change is in comparison to TGplPTH run, which showed a band at only 4.5 kDa. When one of the three components required for the cross-linking reaction (TGplPTH, modified PEG and Factor XIIIa) is missing (i.e. TGplPTH+FXIII or TGplPTH+modified PEG), the band at 4.5 kDa does not change. This gel showed that the TGplPTH reacts quickly and specifically with the modified PEG through the action of Factor XIIIa.

Factor XIIIa-Catalyzed PEGylation is Fast

As judged from the TG-PTH bands, no significant difference between the reaction times (10, 30 and 60 minutes) can be observed. However, the bands presumably corresponding to PEG-modified PTH at higher molecular weight showed an increasing intensity over time, pointing towards a continuation of the reaction up to the 60 minutes time point.

Factor XIIIa Catalyzes Conjugation of TG-PDGF to PEG-VS-PepI

A similar picture emerges with regards to the incorporation of TG-PDGF. SDS-PAGE and silver staining clearly showed that FXIIIa can catalyze the conjugation of TG-PDGF and 8PEG-VS-PepI. Due to the much stronger staining of TG-PDGF, the silver staining reaction was stopped at an earlier time point yielding less background staining of PEG and FXIIIa. Again, the reaction was enabled by Factor XIIIa catalysis and no side reaction involving 8PEG-VS-PepI or Factor XIIIa alone.

In the polyacrylamide gel, TG-PDGF bands at around 35 kDa almost completely disappeared upon reaction with 8PEG-VS-PepI in the presence of Factor XIIIa, suggesting high efficiency of the coupling and bands representing the reaction product appear around 85 kDa and at the top of the gel. This change is in comparison to TGplPDGF run alone (lane TGplPDGF), which shows a band at only 35 kDa. When the enzyme, Factor XIIIa, is missing the band at 35 kDa does not change. From this gel, it can be seen that the TGplPDGF reacts quickly and specifically with the modified PEG through the action of Factor XIIIa.

A time dependence of this reaction was visible with the 10 min reaction time point, showing higher band intensity than the later time points Interestingly, no distinct reaction products (PEGylated PDGF) seemed to be apparent on the stained SDS gel. However, a clearly visible smear between 98 and 188 kDa could be seen that is not present in the control lanes. In light of the broad molecular weight distribution of the 8PEG-VS-PepI itself this may correspond to the PEG-modified growth factor. Since the Lys donor was only used in ca. 7-fold excess over the Gln acceptor on the PDGF the formation of PDGF with more than one PEG bound to it is likely. These multimeric conjugates would show up at very high molecular weights. Indeed, the stained gel shows some bands that apparently did not run through the gel at all due to too high molecular weight.

### Example 2

Two linker molecules, mercaptoethylamine (MEA) and a peptide with the primary sequence AcFKGGERCG (Pep II) (SEQ ID NO: 18), were conjugated to a four arm, endfunctionalized polyethylene glycol tetraacrylate (15 kDa) in a first step to form two precursor components. In a second step, the mercaptoethylamine or peptide modified precursor PEG component was conjugated to a TGplPTH 1-34 (NQEQVSPLYKNR-PTH1-34) (SEQ ID NO: 19) and TGplPDGF.AB (MNQEQVSPLPVELPLIKMPH-PDGF.AB) (SEQ ID NO: 20 to form PEGylated bioactive factors. The conjugation was visualized by silver staining of SDS-PAGE.

Next, the PEGylated bioactive factors were reacted with a second precursor component, a 3.4 kDa polyethylene glycol linear endfunctionalized dithiol and 15 kDa polyethylene glycol tetracacrylate to form a 3-dimensional hydrogel matrix containing the covalently linked bioactive factors. Then, the release of the bioactive factor from the PEG matrices was studied in vitro

1. Coupling of MEA and PepII to 4arm-PEG-Acr via Michael-type addition. PepII and MEA were reacted with PEG-Acr in a 0.6 or 1.2 fold molar excess over acrylate groups in degassed 0.3 M TEA (pH 8.0 at 37° C. for 1 hour). The concentrations of the components are listed in Table 4, further details of the reactions are provided in Table 5.

If all the acrylate groups should have been derivatized by MEA or PepII, the resulting molecule is referred to herein as “PEG-Acr-4MEA”or “PEG-Acr-4PepII”, respectfully. If only two of four acrylate groups are derivatized by MEA or PepII, resulting molecule is referred to herein as “PEG-Acr-2MEA” or “PEG-Acr-2Pep II”, respectfully.

TABLE 5
Reaction scheme to produce PEG-Acr-MEA and PEG-Acr-PepIIP

| | | | | | Re- | |
|---|---|---|---|---|---|---|
| | | | | | action | |
| Product | Ratio | Com- | | Funct. | volume | |
| Name | Linker:Acr | ponents | MW | Groups | (ml) | mM |
| PEG- | 0.6 | MEA | 77.15 | 1 | 1.00 | 48.45 |
| Acr- | | PEG-Acr | 14861 | 4 | 2.00 | 10.09 |
| 2MEA | | | | | | |
| PEG- | 1.2 | MEA | 77.15 | 1 | 1.00 | 96.90 |
| Acr- | | PEG-Acr | 14861 | 4 | 2.00 | 10.09 |
| 4MEA | | | | | | |
| PEG- | 0.6 | PepII | 895 | 1 | 1.00 | 8.07 |
| Acr- | | PEG-Acr | 14861 | 4 | 2.00 | 6.73 |
| 2PepII | | | | | | |
| PEG- | 1.2 | PepII | 895 | 1 | 1.00 | 8.07 |
| Acr- | | PEG-Acr | 14861 | 4 | 2.00 | 13.46 |
| 4PepII | | | | | | |

The thiol content in the reaction was monitored with an Ellman's assay. Therefore, 5 μl of all stock solutions and of the reaction, just after mixing and after completion of the reaction, were shock-frozen. For thiol detection, 20 μl of DNTB-stock solution (0.8 mg/ml) were mixed with 200 μl of reaction buffer (30 mM Tris-HCl, 3 mM ETDA, pH 8.0) and 20 μl of standard or 20 μl of unknown were added (eventually diluted to ca. 0.1-1 mM) and briefly vortexed. 200 μl were pipetted into 96-well plates and absorbance was read at 405 nm with an UV-reader (LMR 1, Lab Exim International). Thiol content was calculated based on a linear regression obtained with cysteine standards ranging from 0.0675 to 1 mM.

The resulting products were subsequently dialyzed (Slide-A-Lyzers, Perbio, MWCO 7000) against ultra pure water for three days at 4° C. After dialysis, the product was lyophilized to obtain a white powder.

2. Factor XIIIa-Catalyzed Coupling of TG-plPTH and plTG-plPDGF into 4PEG-Acr-PepII and 4PEG-Acr-MEA and consequent conjugation into a PEG-matrix

a) Activation of FXIIIa by thrombin. Thrombin was solubilized in 40 mM CaCl₂-solution (500 U/mg final concentration) and 20 μl of thrombin were further diluted with 46.5 μl of CaCl₂-solution. 13.3 μl were added to 200 μl FXIIIa (173 U/ml) and activated for 30 min. at 37° C. Small aliquots (20 μl) of FXIIIa (163 U/ml in 2.5 mM CaCl₂, 4 U/mg thrombin) were stored at −20° C. until further use.

b) Conjugation of PEG-Acr-4MEA and Peg-Acr-4PepII to TG-β/PTH-dansyl. For TG-plPTH-dansyl, the following linking procedure was followed: 10 μl of PEG-Acr-4MEA or PEG-Acr-4PepII (3 mg/ml in 50 mM CaCl₂, 50 mM Tris, pH 7.6) was mixed with 3.5 μl of TG-plPTH-dansyl (1 mg/ml in PBS, pH 7.4) to result in a linker to TG ratio of 7:1. 1.9 μl of activated FXIIIa (diluted to 80 U/ml in Tris) was added after mixing (10 U/ml in reaction). The reaction was carried out at 37° C. and stopped after 10, 30 and 60 min by shock-freezing. Controls of PEG, PTH, FXIIIa, and combinations of each were diluted with the corresponding buffer to result in the same concentration as the samples. All samples were diluted 1:3 with distilled water. SDS-PAGE on 10-20% precast tricine gels (Invitrogen) and silver staining were performed following the manufacturer's protocol. To assure the location of PTH-dansyl on the gel, a dansyl-labeled peptide was used and the gel was visualized by UV-light.

Higher concentrations of PEG-Acr-PepII and TG-plPTH were also tried. While FXIIIa concentration was kept at 10 U/ml in reaction, PEG-Acr-4PepII and TG-plPTH concentration were doubled, tripled and increased tenfold.

c) Conjugation of PEG-Acr-4MEA and PEG-Acr-4PepII with TG-plPDGF. For TG-plPDGF, 10 μl of PEG-Acr-4MEA and PEG-Acr-4PepII (0.580 mg/ml in 3 mM CaCl₂, 50 mM Tris, pH 7.6) were mixed with 4.3 μl of TG-plPDGF (2:8 mg/ml in PBS, pH 7.4) to result in a linker to TG ratio of 7:1. 2.0 μl of activated FXIIIa (diluted to 80 U/ml in Tris) was added after mixing (10 U/ml in reaction). The reaction was carried out at 37° C. and stopped after 10, 30 and 60 min by shock-freezing. Controls of PEG, TG-plPDGF, FXIIIa, and combinations of each were diluted with the corresponding buffer to result in the same concentration as the samples. All samples were diluted 1:7 with distilled water. SDS-PAGE on 10-20% precast tricine gels (Invitrogen) and silver staining were performed following the manufacturer's protocol. Alternatively to silver staining, TG-plPDGF and TG-plPDGF were detected by a PDGF specific Western Blot.

d) Formation of a matrix. The TG-plPTH-dansyl containing matrices were made in a 2-step-reaction. As the PEG-Acr-4PepII had shown the best linking performance (see hereinafter), release studies were done with this linker only.

First, the same reaction as above described for the linking of TG-plPTH to PEG-Acr-4PepII was performed wherby the ratio of PEG-Acr-4 and PepII is chosen such that 50% of the acrylate groups remain unreacted (called PEG-Acr-2PepII). A final TG-piPTH-dansyl concentration of 0.1 mg/ml matrix volume was aimed at. Therefore, 42.2 μl of TG-plPTH-dansyl (1 mg/ml in PBS, pH 7.4) were mixed with a 7-fold excess of PEG-Acr-2-PepII (121 μl, 3.51 mg/ml in 50 mM Tris, 50 mM CaCl₂) and 11 μl FXIIIa (see above, 10 U/ml FXIIIa in reaction). Alternatively, twice as concentrated PEG-Acr-2-PepII was used to achieve a 14-fold excess of lysine over TG-residues. After 1 h of reaction, 6 μl were shock frozen for later gel-electrophoresis. In a consequent second step, the remaining 168 μl were mixed with 150 μl of PEG-Acr (277 mg/ml in 0.3 TEA, pH 7.4) and 150 μl PEG-thiol (141 mg/ml in 0.3 M TEA, pH 7.4) to result in a 1:1 acrylate-thiol ratio and a 7.5% (w/v) PEG-Acr matrix, taking a 10% volume increase by PEG into account. The solution was vortexed for 30 s and 100 μl were pipetted into cut 1 ml syringes. The matrices were weighed and transferred to a release buffer at 37° C. after 1 h.

A control matrix with no FXIIIa was also produced.

For TG-piPDGF, matrices were made similar to the TG-plPTH-dansyl containing matrices described above, with the difference that only 0.01 mg of bidomain bioactive factor/ml matrix volume were incorporated. In a typical recipe, 18.7 μl of PEG-Acr-2PepII (1.03 mg/ml in 50 mM Tris, 3 mM CaCl₂) were mixed with 8.1 μl TG-plPDGF (0.7 mg/ml in PBS) and in a second step 3.8 μl FXIIIa (10 U/ml in final reaction) were added. Alternatively, twice as concentrated PEG-Acr-2PepII was used to achieve a 14-fold excess of lysine over TG-residues. The reaction was performed for 1 h at 37° C. 7.6 μl were removed for SDS-PAGE. In a consequent second step, the rest was mixed with 200 μl PEG-Acr (174.4 mg/ml in 0.3 M TEA, pH 7.4 and 200 μl PEG-thiol (106.1 mg/ml in 0.3 M TEA, pH 7.4). The same procedure as used for the PTH-matrices (described above) was followed after these two steps.

e) Release study. The TG-plPTH-dansyl containing matrices were placed in 1.5 ml PBS and samples were withdrawn after 4 hours and 1, 2, 3, 5 and 7 days and stored at −20° C. until analysis. The buffer was completely exchanged after sampling. The released peptide was measured by means of dansyl fluorescence detection with a Perkin Elmer LS50B luminescence spectrometer at a wavelength of excitation/emission of 330/543 nm. A calibration curve for TG-plPTH-dansyl was obtained by linear regression from samples in the range of 0.75-10 μg/1 TG-plPTH-dansyl.

The TG-plPDGF containing matrices were placed in 10 μl PBS (10 mM, pH 7.4 containing 0.1% bovine serum albumin) at 37° C. and samples of 150 μl withdrawn after 4 hours and 1, 2, 3 and 5 days and stored at −20° C. until analysis. The samples were diluted 40 times with TBS, 0.1% BSA and were analyzed by an ELISA specific for TG-plPDGF-AB.

Results and Discussion

Production of PEG-Acr-2MEA and PEG-Acr-2PepII, PEG-Acr-4MEA and PEG-Acr-4PepII

4-arm-PEG-Acr was functionalized to obtain both fully amine derivatized PEG-Acr as well as 4-arm-PEG with two amine and two acrylate groups on average. The reaction of PEG-Acr with the thiol-residue of MEA or PepIl by means of Michael-type addition proceeded very fast at pH 8.0 (in the order of minutes). Theoretical starting and end thiol concentrations were in good agreement with measured values in case of MEA. In case of the peptide, complete disappearance of thiols was seen when a 1.2 thiol-acrylate ratio was employed, indicating that disulfide-formation had occurred to a minor degree (possibly already in the starting material). It was nevertheless assumed that a functionalization of close to 50 and 100%, respectively, was achieved (Table 6).

TABLE 6
PEG-Acr-Linker production: expected and
measured thiol concentrations

| Ratio | Ex- | | | | Ex- |
|---|---|---|---|---|---|
| Linker/ | pected | 0 min | 1 min | 60 min | pected |
| Arc | Start | (measured) | (measured) | (measured) | Finish |
| MEA 1.2 | 32.0 | 37.0 | 6.3 | 4.1 | 5.3 |
| MEA 0.6 | 16.0 | 19.6 | −0.3 | −0.3 | 0.0 |
| PepII 1.2 | 5.4 | 3.5 | 0.6 | 0.0 | 0.0 |
| PepII 0.6 | 6.5 | 4.2 | 0.6 | 0.1 | 1.1 |

PEGylation of TG-plPTH-Dansyl and Matrix Formation

PEG-Acr-4PepII. SDS-PAGE with fluorescence detection and consequent silver staining allowed clear location of the TG-plPTH-dansyl on the gel and determination of its MW. When PEG-Acr-PepII, TG-plPTH-dansyl and FXIIIa were reacted, the band at 5 kDa became weaker and a new broad band appeared at ca. 40 kDa which was fluorescent. As PEG has a larger radius of gyration than proteins, it can be expected to appear at a higher MW than 15 kDa. Therefore, the fluorescent band at 40 kDa was determined to be PEG-TG-PLPTH-dansyl. The band did not appear when FXIIIa was not added, proving the FXIIIa-dependence of the reaction. Quantification of the linking was difficult, however. By comparing the band intensities an estimate that 50-80% of TG-plPTH had reacted. Compared to results obtained with PEG-VS-PepI, the derivatization is less complete, indicating that a spacer between the cysteine and the lysine group might be beneficial for the reaction.

Mercaptoethylamine-linker. Like for PEG-Acr-PepII, a band at ca. 40 kDa appeared when PEG-Acr-MEA was reacted with TG-plPTH-dansyl in presence of FXIIIa. This band was however much weaker (10-20% of what was seen for PEG-Acr-PepII). Thus, it appeared that affinity of FXIIIa towards an ethylamine was lower than for a butylamine as it is present in lysine.

Matrix formation and retention of PTH-dansyl. In order to achieve a high TG-plPTH-dansyl concentration (1 mg/ml matrix), the linking experiments were performed at 3 and 10 times higher concentration of TG-plPTH-dansyl than in the previous experiments. However, already at a threefold and especially at a tenfold concentration, precipitation of TG-plPTH-dansyl occurred in presence of PEG and the consequent linking was not successful.

Therefore, the original conjugation recipe was only slightly adapted and matrices containing 0.1 mg TG-plPTH-dansyl per ml matrix were produced. SDS-PAGE and detection of the peptide by fluorescence and silver staining confirmed that TG-plPTH-dansyl had been linked to PEG-Acr-2PepII. The two remaining acrylate groups of the PEG-Pep-TG-plPTH-conjugate could be covalently linked into a PEG-matrix by Michael-type addition of PEG-dithiol.

The release profile of TG-plPTH-dansyl clearly confirmed the successful linking and consequent cross-linking into the matrix. In absence of FXIIIa only 7% of TG-plPTH-dansyl were retained more than 5 days (168 hours) compared to 63% retention in a matrix where a 7-fold excess of PEG-peptide groups over TG-plPTH was employed and a 88% in case of a 14-fold excess (see Table 7).

TABLE 7
Retention of TG-plPTH-dansyl in % from PEG-matrices
as measured by dansyl-fluorescence

| | No FXIIIa | 7-fold excess of PEG- | 14 fold excess of |
|---|---|---|---|
| | added (% TG- | Acr-PepII over TG- | PEG-Acr-PepII over |
| | plPTH-dansyl | PTH-dansyl | TG-PTH-dansyl |
| Time | retained | (% TG-plPTH-dansyl | (% TG-plPTH-dansyl |
| (hours) | in matrix) | retained in matrix) | retained in matrix) |
| 0 | 100 | 100 | 100 |
| 4 | 74 | 96 | 92 |
| 24 | 47 | 86 | 88 |
| 48 | 40 | 81 | 88 |
| 72 | 32 | 75 | 88 |
| 120 | 19 | 68 | 88 |
| 168 | 7 | 63 | 88 |

PEGylation of TG-plPDGF to PEG-Acr-PepII and Matrix Formation

For TG-plPDGF, just the PEG-Acr-PepII-linker was tested as it was most successful for TG-plPTH. SDS-PAGE and silver staining showed a partial disappearance of TG-plPDGF (dimer running at 35 kDa). A new band could be identified by Western Blot in the form of a smear ranging between 50 and 90 kDa, with stronger bands at around 50, 60 and 70 kDa which did not appear when FXIIIa was missing in the reaction or when FXIIIa was mixed with TG-plPDGF only. As TG-plPDGF has two TG-sites, a protein that is linked to two PEGs or, as each PEG carries an average of two lysines, PEG with multiple TG-plPDGF can be formed. All of these reactions would result in different MWs, which is probably why several bands were present. Comparing the band intensity of TG-plPDGF at 35 kDa with standards of 100, 33 and 10% TG-plPDGF, we estimate that more than 70% of TG-plPDGF was linked to PEG-Acr-4PepII.

Matrices were made in a 2-step-reaction, the first step corresponding to the previous linking experiments with the difference that a bifunctional PEG was used (containing two peptides and two acrylate groups, named PEG-Acr-2PepII). From this reaction samples were withdrawn and run with additional standards over an SDS-gel. Silver staining showed that when PEG-Acr-2PepII was reacted with TG-plPDGF in presence of FXIIIa, the band-intensity at 35 kDa was reduced by ca. 50-60% in case a 7 fold excess of PEG-Acr-2PepII over TG-as plPDGF was employed (as judged by visual comparison with TG-plPDGF standards). When a 14-fold excess was employed, the band intensity reduction was only little more pronounced. It might be that at higher PEG-concentrations the more favorable amine-donor ratio is out leveled by more PEG disturbing the reaction.

The reaction was clearly FXIIIa dependent as no band shift was seen when FXIIIa was missing.

The successful linking was confirmed with release experiments where TG-plPDGF appearing in a release buffer was measured by an ELISA-assay. While in absence of FXIIIa only 4% of TG-plPDGF was retained in the matrix for more than 5 days, 47% was retained when FXIIIa was employed in the reaction with a 7-fold excess of lysine-groups over TG-sites and even 54% was retained with a 14-fold excess.

TABLE 8
Retention of TG-plPDGF in % from PEG-matrices as measured by
ELISA

| | | PEG-Acr-2PepII | PEG-Acr-2PepII |
|---|---|---|---|
| | | 7-fold excess | 14-fold excess |
| | No FXIIIa Added | compared to TG- | compared to TG- |
| | (% TG-plPDGF | plPDGF | plPDGF |
| Time | retained in | (% TG-plPDGF | (% TG-plPDGF |
| (hr) | matrix) | retained in matrix) | retained in matrix) |
| 0 | 0 | 100 | 100 |
| 4 | 61 | 75 | 85 |
| 24 | 37 | 68 | 71 |
| 48 | 21 | 53 | 65 |
| 72 | 13 | 52 | 63 |
| 120 | 4 | 47 | 54 |

Those skilled in the art will recognize, or be able to ascertain using no more than routine experimentation, many equivalents to the specific embodiments of the invention described herein. Such equivalents are intended to be encompassed by the following claims.

We claim:

1. A synthetic precursor component or a synthetic biomaterial comprising a bioactive factor or bidomain bioactive factor, wherein the bioactive factor or the bidomain bioactive factor is covalently bound to the precursor component or biomaterial by an enzymatically degradable linkage.

2. The synthetic precursor component or synthetic biomaterial of claim 1, wherein the bidomain bioactive factor comprises a first and a second domain, wherein the first domain comprises a substrate domain for cross-linkable enzymes and the second domain comprises a bioactive factor.

3. The synthetic precursor component or synthetic biomaterial of claim 2 wherein the substrate domain for cross-linkable enzymes is a tissue transglutaminase substrate domain.

4. The synthetic precursor component or synthetic biomaterial of claim 3 wherein the tissue transglutaminase substrate domain is a Factor XIIIa substrate domain.

5. The synthetic precursor component or synthetic biomaterial of claim 2 wherein the bioactive factor is selected from the group comprising of small molecules, hormones, nucleotides, peptides, and proteins.

6. The synthetic precursor component or synthetic biomaterial of claim 5 wherein the bioactive factor is selected from the group consisting of parathyroid hormone (PTH), platelet-derived growth factor (PDGF), transforming growth factor betas (TGF β), bone morphogenetic protein (BMP) insulin-like growth factors (IGF), fibroblast growth factors (FGF).

7. The synthetic precursor component or synthetic biomaterial of claim 1, comprising a polyethylene glycol.

8. A method of forming a synthetic biomaterial comprising at least one bioactive factor covalently linked to the biomaterial, comprising catalyzing the formation of the covalent linkage using at least one enzyme.

9. The method of claim 8, wherein the enzyme is a tissue transglutaminase.

10. The method of claim 9, wherein the tissue transglutaminase is Factor XIIIa.

11. The method of claim 8, wherein the bioactive factor is a bidomain bioactive factor comprising a first and a second domain wherein the first domain comprises a substrate domain for a crosslinking enzyme and the second domain comprises the bioactive factor.

12. The method of claim 11, wherein the first domain is a Factor XIIIa substrate domain.

13. The method of claim 8, further comprising forming the biomaterial from at least two precursor components using a Michael type addition reaction, wherein the first precursor component comprises n nucleophilic groups and the second precursor component comprises m electrophilic groups, wherein n and m are at least two and the sum n+m is at least five.

14. The method of claim 13, wherein the nucleophilic groups comprise thiol groups.

15. The method of claim 13, wherein the electrophilic groups comprise conjugated unsaturated groups.

16. The method of claim 13, wherein the bioactive factor is a bidomain bioactive factor comprising a first and a second domain, wherein the first domain comprises a substrate domain for a crosslinking enzyme and the second domain comprises the bioactive factor, and
wherein at least one of the precursor components further comprises at least one amine group,
the method further comprising reacting via enzymatic catalysis at least one amine group on at least one of the precursor components with the first domain of the bidomain bioactive factor.

17. The method of claim 16, wherein the second precursor component comprises at least one amine group.

18. The method of claim 17, further comprising forming the second precursor component by reacting a precursor component with a linker molecule having a formula selected from the group consisting of HS—(X)ₙ—NH₂ and HS—(Xᵢ)ₙ—NH₂, wherein X is any suitable group.

19. The method of claim 18, wherein HS—(X)ₙ—NH₂ is mercaptoethylamine.

20. The method of claim 13, wherein at least one precursor component comprises a polyethyleneglycol.

21. The method of claim 8, wherein the bioactive factor is selected from the group comprising of small molecules, hormones, nucleotides, peptides, and proteins.

22. The method of claim 21, wherein the bioactive factor is selected from the group consisting of parathyroid hormone (PTH), platelet-derived growth factor (PDGF), transforming growth factor betas (TGF β), bone morphogenetic protein (BMP) insulin-like growth factors (IGF), fibroblast growth factors.

23. A synthetic biomaterial comprising a bidomain bioactive factor or bioactive factor covalently bound thereto wherein the bioactive factor or bidomain bioactive factor is covalently bound to the biomaterial by enzymatic catalysis.

24. The biomaterial of claim 23, wherein the biomaterial is formed from at least two precursor components, wherein the first precursor component comprises n nucleophilic groups and the second precursor component comprises m electrophilic groups, wherein n and m are at least two and the sum n+m is at least five, and wherein the first precursor and the second precursor are capable of undergoing a Michael type addition reaction.

25. The biomaterial of claim 24, wherein the nucleophilic groups comprise thiol groups.

26. The biomaterial of claim 24, wherein the electrophilic groups comprise conjugated unsaturated groups.

27. The biomaterial of claim 24, wherein the bidomain bioactive factor comprises a first and a second domain, wherein the first domain comprises a substrate domain for cross-linkable enzymes and the second domain comprises a bioactive factor.

28. The biomaterial of claim 27, wherein the first domain is a Factor XIIIa substrate domain.

29. The biomaterial of claim 24, wherein at least one of the precursor components further comprises at least one amine group capable of reacting with the first domain of the bidomain bioactive factor or the bioactive factor under enzymatic catalysis.

30. The biomaterial of claim 29, wherein the second precursor component comprises at least one amine group.

31. The biomaterial of claim 23, wherein the bioactive factor is selected from the group comprising of small molecules, hormones, nucleotides, peptides, and proteins.

32. The biomaterial of claim 31, wherein the bioactive factor is selected from the group consisting of parathyroid hormone (PTH), platelet-derived growth factor (PDGF), transforming growth factor betas (TGF β), bone morphogenetic protein (BMP) insulin-like growth factors (IGF), fibroblast growth factors (FGF).

33. The biomaterial of claim 24, wherein the precursor component comprises a polyethylene glycol.


SEQUENCE LISTING (20 sequences)

SEQ ID NO: 1 - protein, 5 aa, Homo sapiens
YIGSR

SEQ ID NO: 2 - protein, 5 aa, Homo sapiens
GAKDV

SEQ ID NO: 3 - protein, 4 aa, Homo sapiens
KKKK

SEQ ID NO: 4 - protein, 8 aa, Homo sapiens
NQEQVSPL

SEQ ID NO: 5 - protein, 16 aa, Homo sapiens
YRGDTIGEGQQHHLGG

SEQ ID NO: 6 - protein, 5 aa, Homo sapiens
GYKNR

SEQ ID NO: 7 - protein, 6 aa, Homo sapiens
LIKMKP

SEQ ID NO: 8 - protein, 6 aa, Homo sapiens
NFKSQL

SEQ ID NO: 9 - protein, 8 aa, Homo sapiens
  Acetylated (1)..(1)
GPLALTAL

SEQ ID NO: 10 - protein, 6 aa, Homo sapiens
  Acetylated (1)..(1)
PFELRA

SEQ ID NO: 11 - protein, 5 aa, Homo sapiens
ZAAFA

SEQ ID NO: 12 - protein, 8 aa, Homo sapiens
GPLGIAGP

SEQ ID NO: 13 - protein, 8 aa, Homo sapiens
PHYGRSGG

SEQ ID NO: 14 - protein, 9 aa, Homo sapiens
PGSGRSASG

SEQ ID NO: 15 - protein, 4 aa, Homo sapiens
CRGD

SEQ ID NO: 16 - protein, 4 aa, Homo sapiens
CGKG

SEQ ID NO: 17 - protein, 16 aa, Artificial sequence
  Bifunctional peptide linker molecule
FKGGGPQGIWGQERCG

SEQ ID NO: 18 - protein, 8 aa, Artificial sequence
  Linker molecule
FKGGERCG

SEQ ID NO: 19 - protein, 12 aa, Artificial sequence
  plasmin degradable sequence and Factor XIIIa substrate domain
NQEQVSPLYKNR

SEQ ID NO: 20 - protein, 20 aa, Artificial sequence
  Transglutaminase and plasmin degradable sequences
MNQEQVSPLPVELPLIKMPH